(19) United States
(12) Patent Application Publication
(10) Pub. No.: US 2016/0376159 A1
(43) Pub. Date: Dec. 29, 2016

(54) ZEOLITES WITH MWW FRAMEWORK FORMED OF SILICON AND ZINC OR IRON, AND RELATED METHODS

(71) Applicant: AGENCY FOR SCIENCE, TECHNOLOGY AND RESEARCH, Singapore (SG)

(72) Inventors: Yan LIU, Singapore (SG); Armando BORGNA, Singapore (SG)

(21) Appl. No.: 15/125,135

(22) PCT Filed: Mar. 10, 2015
(86) PCT No.: PCT/SG2015/050034

§ 371 Date: Sep. 9, 2016

(30) Foreign Application Priority Data
Mar. 10, 2014 (SG) 10201400517T

(51) Int. Cl. C01B 39/48; B01J 29/88; C07C 5/48; B01J 37/10; B01J 37/00; C07C 45/00; C01B 39/08; B01J 29/04

(52) U.S. Cl. CPC C01B 39/48; C01B 39/08; C01B 39/087; B01J 29/88; B01J 29/048; B01J 37/10; B01J 37/0018; C07C 45/002; C07C 5/48; C01P 2002/72; C01P 2002/82; C07C 2529/04

(57) ABSTRACT

A Zn—Si or Fe—Si zeolite having an MWW framework may be formed by treating a gel solution with a hydrothermal treatment. The gel solution includes water, a silicon source, a zinc or iron source, and an MWW-templating agent. The hydrothermal treatment may include dissolving the source materials in the gel solution, and heating the gel solution with the dissolved sources to induce hydrothermal crystallization in the gel solution. The zeolite can be substantially free of aluminum, and can be used in a catalyst, or in a separation or purification device.

### RELATED APPLICATIONS

This application claims the benefit of and priority from Singaporean Patent Application No. 10201400517T, filed Mar. 10, 2014, the entire contents of which are incorporated herein by reference.

### FIELD

The present invention relates generally to zeolites and their preparation, and particularly to zeolites having a MWW framework, their preparation and uses, and materials and devices including such zeolites.

### BACKGROUND

Zeolites containing aluminum (Al) and silicon (Si) in their zeolite frameworks are known. Many known zeolites are generally microporous aluminosilicate materials, which include TO₄ tetrahedral structural units, where T is aluminum (Al³⁺) or Si⁴⁺. Some of these zeolites have exhibited useful catalytic and adsorption properties. It has also been shown that introducing different heteroatoms into the framework of the zeolites may lead to different catalytic and adsorption properties. For example, it is known that titanium-silicates are efficient for hydroxylation of aromatics, whereas iron-zeolites are effective catalysts for redox reaction. Gallosilicate has been used for the aromatization of light paraffin.

Some zeolites have an MWW framework formed of Si and Al. Zeolites with an MWW framework include MCM-22, MCM-49, MCM-56, and MCM-36 zeolites, and the like. Some MCM-22 zeolites have been shown to have good catalytic performance on many different reactions, including alkylation, aromatization, disproportionation of toluene, catalytic cracking, and the like, The MCM-22 framework has been shown to have layers linked together along the c-axis by oxygen bridges and contains two independent pore systems, which are accessible through 10-MR (Membered Ring) ring apertures. One of the two pore systems has two-dimensional sinusoidal 10-MR ring channels, and the other pore system has large super cages with inner dimensions of 7.1×7.1×18.2 Å, characterized by 12-MR ring pockets located on the external surface. It is expected that the 12-MR ring pockets _{playa} significant role in many catalytic reactions as they can accommodate larger organic molecules than 10-MR ring apertures can do, due to MCM-22's porous structure, high microporousity, high hydrothermal stability and mild acid property,

### SUMMARY

In one aspect, the present disclosure relates to a method, in which a gel solution is subjected to a hydrothermal treatment, to form a Zn—Si (also referred to as ZS-1) or Fe—Si zeolite having an MWW framework. The gel solution comprises water, a silicon source, a zinc or iron source, and an MWW-templating agent. The gel solution may be maintained at a first temperature to dissolve the silicon source and the zinc or iron source in the gel solution. The hydrothermal treatment may comprise heating the gel solution with the dissolved silicon source and zinc or iron source at a second temperature above the first temperature, to induce hydrothermal crystallization in the gel solution, thus forming a crystalline Zn—Si or Fe—Si zeolite having the MWW framework. The crystalline zeolite material may be calcined. The first temperature may be in the range from room temperature to about 70° C. and the second temperature may be in the range from about 140° C. to about 160° C. The calcination temperature may be about 550° C. The gel solution may be maintained at the first temperature for a period of up to about 24 hours, and heated at the second temperature for a period of 1 to 6 weeks. The MWW-templating agent may comprise a hexamethyleneimine. The silicon source may be selected from colloidal silica, fumed silica, silicon oxides, silicates, and mixtures thereof. The zinc or iron source may be selected from zinc nitrates, zinc halides, zinc sulfates, zinc carbonates, zinc alkoxides, and mixtures thereof. A molar ratio of the silicon source to the zinc or iron source in the gel solution may be from 7:1 to 20:1. The gel solution may comprise about 20 wt % of the silicon source and about 3 to about 4 wt % of the zinc or iron source, based on a total weight of the gel solution. The gel solution may comprise a base. The base may comprise sodium hydroxide. The gel solution may have a pH value of above 7 before the hydrothermal treatment. The gel solution may comprise seeds having an MWW framework. The MWW framework may be substantially free of aluminum. The zeolite may be a Zn—Si zeolite when the zinc or iron source is a zinc source. The zeolite may be a Fe—Si zeolite when the zinc or iron source is an iron source. The zeolite may be characterized by an x-ray diffraction pattern comprising the peaks listed in a table or figure disclosed herein.

In another aspect, the present disclosure relates to a zeolite prepared according to a method disclosed herein.

In a further aspect, the present disclosure relates to a zeolite Zn—Si zeolite having an MWW framework substantially free of aluminum.

In another aspect, the present disclosure relates to a zeolite disclosed herein and characterized by an x-ray diffraction pattern comprising the peaks shown in any one of Table I, Table III or FIGS. 1A, 1B.

In a further aspect, the present disclosure relates to a Fe—Si zeolite having an MWW framework substantially free of aluminum.

In another aspect, the present disclosure relates to a zeolite disclosed herein and characterized by an x-ray diffraction pattern comprising the peaks shown in any one of Table II, Table IV, or FIG. 4.

In a further aspect, the present disclosure relates to a catalyst comprising a zeolite disclosed herein. The catalyst may be used in catalyzing an activation, oxidehydrogenation, or dehydrogenation reaction.

In another aspect, the present disclosure relates to a separation or purification device comprising a zeolite disclosed herein.

Other aspects, features, and embodiments of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention:

FIGS. 1A and 1B are line graphs showing x-ray diffraction (XRD) patterns of sample ZS-1 zeolite, illustrative of an embodiment of the present invention;

FIG. 2 is a line graph showing Fourier transform infrared spectroscopy (FTIR) spectra of the sample zeolite of FIGS. 1A, 1B and comparison zeolites;

FIG. 3 is a line graph showing Raman spectra of the sample zeolite of FIGS. 1A, 1B and comparison zeolites;

FIG. 4 is a line graph showing an XRD spectrum of a sample Fe—Si zeolite, illustrative of an embodiment of the present invention;

FIG. 5 is a data graph showing representative activity data for ethanol dehydrogenation with a sample catalyst;

FIG. 6 is a data graph showing representative activity data for butane dehydrogenation with sample catalysts;

FIG. 7 is a data graph showing representative catalytic activity for butane dehydrogenation with a comparison catalyst;

FIG. 8 is a data graph showing representative stability data for butane conversion with sample catalysts

FIG. 9 is a data graph showing representative stability data for butane conversion with a comparison catalyst;

FIG. 10 is a bar graph showing representative selectivity data for a sample catalyst;

FIG. 11 is a data graph showing representative catalytic activity of a sample catalyst for different reactions; and

FIG. 12 is a schematic three-dimensional representation of an MWW framework in a zeolite.

### DETAILED DESCRIPTION

An embodiment of the present disclosure relates to zeolites having an MWW framework formed of zinc and silicon, where the MWW framework is substantially free of aluminium. These zeolites are referred to as Zn—Si zeolite with MWW framework or “ZS-1” herein.

In an embodiment disclosed herein, a ZS-1 zeolite is prepared by hydrothermally treating a precursor gel solution. The gel solution includes water, a silicon (Si) source (or precursor), a zinc (Zn) source (or precursor), and an MWW-templating agent. The gel solution is free of aluminum.

The gel solution is initially maintained at a first temperature suitable to dissolve the Si and Zn sources or precursors in the gel solution. The gel solution is then heated at a second temperature above the first temperature to crystalize the material for forming a ZS-1 zeolite. The crystalized material may he heated to a calcination temperature, such as about 550° C. for 7 to 12 hours, to decompose or remove undesired residues from the zeolite structure.

The reaction conditions including the reaction temperatures, reaction pressure, and the contents of in the gel solution are selected to induce hydrothermal crystallization. The MWW-templating agent may be hexamethyleneimine (HMI).

For example, in selected embodiments, the gel solution may be heated in an autoclave at an autogenous pressure. The first temperature may be from room temperature to about 70° C. The second temperature may be from about 140° C. to about 160° C. The gel solution may be maintained at the first temperature for a period of up to about 24 hours. The gel solution may be heated at the second temperature for a period of 1 to 6 weeks.

In different embodiments, the Si source may be selected from colloidal silica, fumed silica, silicon oxides such as silicon alkoxides, silicates, and mixtures thereof. Suitable Si sources may also include other Si containing materials.

In different embodiments, the Zn source may be selected from zinc nitrates, zinc halides, zinc sulfates, zinc carbonates, zinc alkoxides, and mixtures thereof. Suitable Zn sources may also include other Zn containing materials.

The molar ratio of the Si precursor to the Zn precursor in the gel solution may be from 7:1 to 20:1. The weight ratio of the Si precursor to the Zn precursor in the gel solution may be from 2:1 to 15:1. For example, the gel solution may contain about 20 wt % of the Si source and about 3 wt % of the Zn source, based on the total weight of the gel solution.

In some embodiments, the gel solution may also contain a base, such as sodium hydroxide.

In alternative embodiments, the gel solution may also contain seeds with MWW framework, also referred to as MWW-templating agent.

The gel solution may have a pH above 7 before heating.

The precursor materials used in the hydrothermal treatment may be prepared according to, and the treatment process may be modified from, known hydrothermal processes and known processes for preparing a particular raw or precursor material. For example, materials and processes disclosed in the following references may be adapted for use in an embodiment disclosed herein: U.S. Pat. No. 4,954,325 to Rubin et al.; Lawton et al., J. Phys. Chem. 100 (1996) 3788; Corma et al., Zeolites 15 (1995) 2; Ravishankar et al., Stud. Surf. Sci. Catal. 84 (1994) 331; Mochida et al., Zeolites 18 (1997) 142; Guray et al., Micropor. Mesopor. Mater. 31 (1999) 241; Marques et al, Micropor. Mesopor. Mater. 32 (1999) 131; Cheng et al., Micropor. Mesopor. Mater. 42 (2001) 307-316; and Sha et al., Material Transactions 46 (2005) 2651-58, the contents of each of which are incorporated herein by reference.

Further information for forming MWW seeds or templates, and for processes related to other types of zeolites can be found in the following references. He et al., “From Zeolites to Porous MOF Materials—the 40th Anniversary of International Zeolite Conference,” R. Xu, Z. Gao, J. Chen, W. Yan (Editors), 2007, 470; Kumar et al., Appl. Catal. A 147 (1996) 175; Nagy et al., Mol. Sieves 5 (2007) 365; Wu et al., Chin. J. Catal. 25 (2004) 427; Liu et al., Shiyou Xuebao, Shiyou Jiagong 22 (2006) 198; U.S. Pat. No. 7,186,872; WO 03/074422 A1; CN1353011 (A); DE19939416 (Al); CN100453178; Gabrienko et al., J. Phys. Chem. C 114 (2010) 12681; Kolyaqin et al., J. Phys. Chem. C 112 (2008) 20065; Stepanov et al., ChemPhysChem 9 (2008) 2559; CN 101391177A; and Folie et al., Ind. Eng. Chem. Res. 47 (2008) 1501, the contents of each of which are incorporated herein by reference.

Conveniently, a pure ZS-1 zeolite can be formed with a process disclosed herein, where the MWW framework is free of aluminum (Al). The ZS-1 zeolite may be a pure Zn—Si zeolite.

In a particular embodiment, appropriate amounts of sodium hydroxide and a zinc source such as zinc nitrate hexahydrate may be mixed and dissolved in water, such as distilled water, to form a solution. The molar ratio of sodium hydroxide to zinc nitrate in the solution may be varied from about 1 to about 5. A suitable amount of hexamethyleneimine may be added to the solution as a template agent and the mixture may be agitated or stirred for a period of time to mix the agents well. The amount of hexamethyleneimine in the mixture may be such that the molar ratio of hexamethyleneimine to the zinc source is about 2 to about 10.

A silica precursor such as colloidal silica and a seeding agent such as MCM-22 (MWW) seeds may be added to the mixture to form a gel. The colloidal silica may be LUDOX™ HS-40 colloidal silica available from Sigma Aldrich™. The MCM-22 seeds may be seeds formed of calcined or uncalcined sodium MWW. The weight ratio of silica to the seeds may be from about 100 to about 200, such as about 150. The weight ratio of silica to the zinc source may be about 5. The resulting mixture may contain about 16 wt % of silica and about 3 wt % of the zinc source. The resulting mixture is agitated or stirred to allow mixing and reaction of the reagents, for a suitable period of time such as about four hours until the gel is formed.

The resulting gel solution is hydrothermally crystalized at an elevated temperature. The hydrothermal treatment may be carried out in an autoclave. The gel may be heated to a suitable temperature for a period of time sufficient to crystallize it. For example, the autoclave containing the gel solution may be heated in a rotary oven at about 150° C. for 2 to 6 weeks.

The crystalized material can be cooled and separated out, and can be cleaned such as washed thoroughly with distilled water. The cleaned material is then dried, such as at 80° C. to 110° C. for several hours.

The dried material is calcined, such as in a static air furnace, at 550° C. for 7 to 12 hours. The resulting product is a ZS-1 zeolite material with a MWW framework.

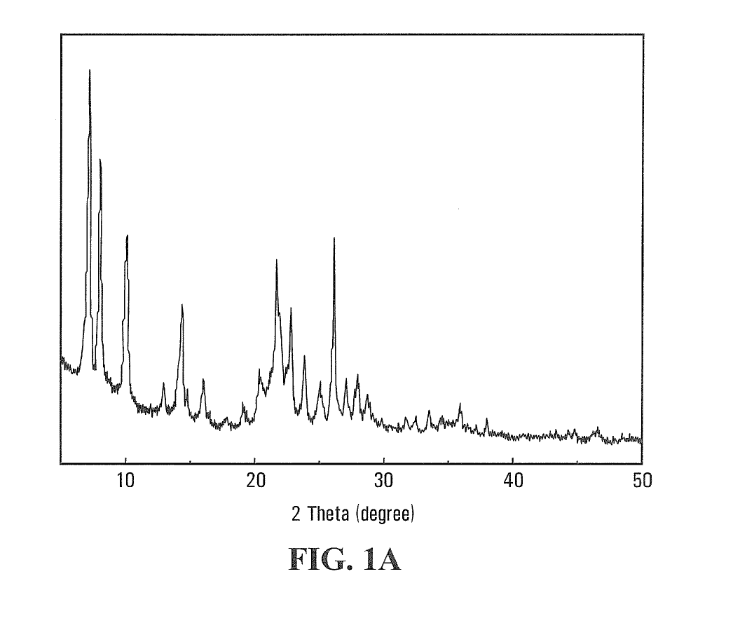

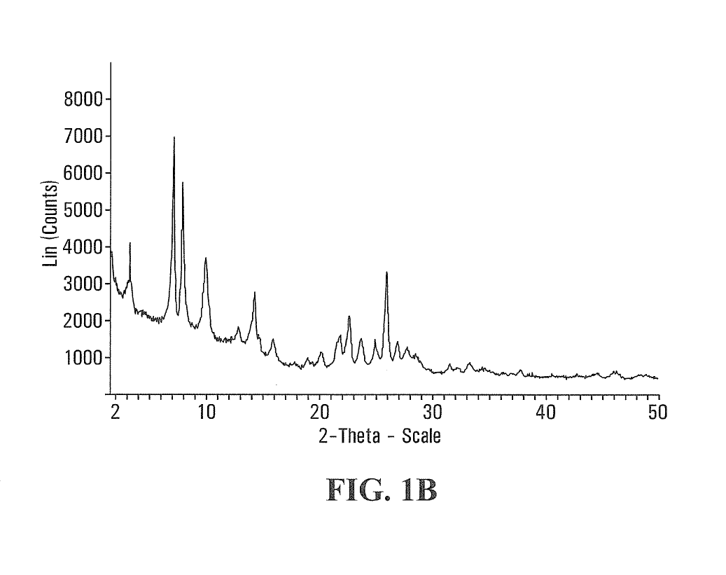

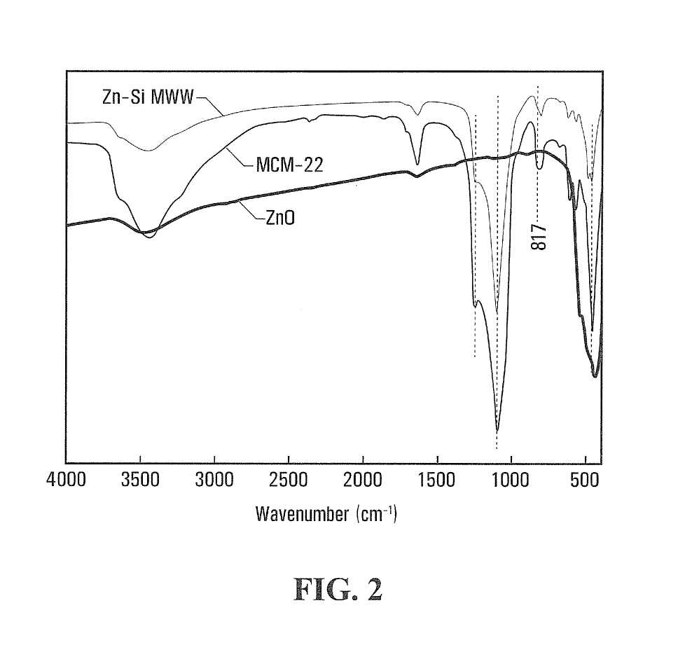

A calcined ZS-1 zeolite may be characterized by the XRD (x-ray diffraction) spectrum or pattern shown in FIGS. 1A, 1B, or the Fourier transform infrared spectroscopy (FTIR) spectrum shown in FIG. 2, or by both. In particular, a ZS-1 zeolite may be characterized by the 2-θ peaks listed in Table I.

TABLE I
XRD peaks of ZS-1 Zeolite

| Peak (d-value) | 2-θ (degree) | Relative Intensity |
|---|---|---|
| 12.4 | 7.2 | High |
| 11.1 | 8.0 | High |
| 8.85 | 10.0 | Medium |
| 6.17 | 14.3 | Medium |
| 3.91 | 22.7 | Medium |
| 3.42 | 26.1 | Medium |

The present disclosure further relates to a catalyst comprising a ZS-1 zeolite was disclosed herein. Such a catalyst may provide unusual oxidehydrogenation properties. Thus, this catalyst may be useful in activation or reactions involving small alkanes, alkenes, alcohols and related compounds. Typical examples of reactions in which the catalyst may be used include alcohol oxidehydrogenation to oxygenates, and hydrocarbons dehydrogenation or oxidehydrogenation to olefins and dienes. For instance, a ZS-1 zeolite disclosed herein may have catalytic activity for direct ethanol dehydrogenation, or for butene oxidative dehydrogenation.

The present disclosure also relates to a device for separating or purifying materials, wherein the device comprises a ZS-1 zeolite. For example, the device may be used for purifying a gas or separating certain substances from an input gas. It may be expected that such a device may be used to purify gases containing low concentrations of organic compounds, or to remove phosphine.

In another aspect of the disclosure, it is expected that Zn in the above description may be replaced with iron (Fe), and Fe—Si zeolites with an MWW structure, similar to the Zn—Si zeolites described above, may be formed in a similar process.

For example, in a particular embodiment, appropriate amounts of sodium hydroxide and an iron source such as iron nitrate hydrate may be mixed and dissolved in water, such as distilled water, to form a solution. The molar ratio of sodium hydroxide to iron nitrate hydrate in the solution may be about 7:1 to 15:1, corresponding to a weight ratio of about 0.22. A suitable amount of hexamethyleneimine may be added to the solution and the mixture may be agitated or stirred for a period of time to mix the agents well. The amount of hexamethyleneimine in the mixture may be such that the molar ratio of hexamethyleneimine to the iron source is about 2 to about 10.

A silica precursor such as colloidal silica and a seeding agent such as MCM-22 (MWW) seeds may be added to the mixture to form a gel. The colloidal silica may be LUDOX™ HS-40 colloidal silica available from Sigma Aldrich™. The MCM-22 seeds may be seeds formed of calcined or uncalcined sodium MWW. The weight ratio of silica to the seeds may be from about 100 to about 200, such as about 150. The weight ratio of silica to the iron source may be about 3.8. The resulting mixture may contain about 16 wt % of silica and about 4 wt % of the iron source. The resulting mixture is agitated or stirred to allow mixing and reaction of the reagents, for a suitable period of time such as about four hours until the gel is formed.

The resulting gel solution is hydrothermally crystalized at an elevated temperature. The hydrothermal treatment may be carried out in an autoclave. The gel may be heated to a suitable temperature for a period of time sufficient to crystallize it. For example, the autoclave containing the gel solution may be heated in a rotary oven at about 150° C. for 2 to 6 weeks.

The crystalized material can be cooled and separated out, and can be cleaned such as washed thoroughly with distilled water. The cleaned material is then dried, such as at 80° C. to 110° C. for several hours.

The dried material is calcined, such as in a static air furnace, at 550° C. for 7 to 12 hours. The resulting product is a Fe—Si zeolite material with a MWW framework.

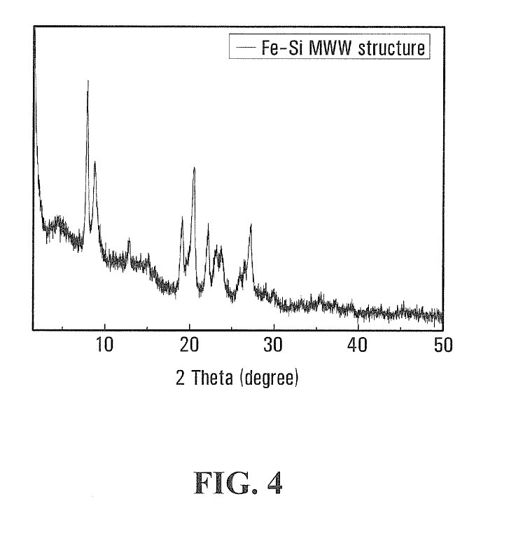

A Fe—Si zeolite with MWW framework as disclosed herein may be characterized by the XRD peaks shown in FIG. 4, or the peaks listed in Table II.

TABLE II
XRD peaks of Fe—Si Zeolite with MWW framework

| Peak (d-value) | 2-θ (degree) | Relative Intensity |
|---|---|---|
| 11.2 | 7.9 | High |
| 10.1 | 8.8 | Medium |
| 6.87 | 12.9 | Low |
| 5.85 | 15.1 | Low |
| 4.00 | 22.2 | Medium |
| 3.35 | 26.6 | Low |

Selected embodiments of the present disclosure may be used in selective adsorption and catalytic applications. For example, a zeolite disclosed herein may be used as a catalyst for small alkanes activation and reaction. A zeolite disclosed herein may also be useful for catalyzing reactions such as alkylation, aromatization, disproportionation of toluene, catalytic cracking, or the like

Also, a zeolite disclosed herein may be potentially used for purifying a gas containing a low-concentration of organic compounds, or for phosphine removal, or the like. It may be used as an absorbent for waste gases.

Other features, modifications, and applications of the embodiments described here may be understood by those skilled in the art in view of the disclosure herein.

The following examples are provided to further illustrate embodiments of the present invention, and are not intended to limit the scope of the disclosure.

### EXAMPLES

### Example I

### Synthesis and Characterization of Sample ZS-1 Zeolite

Sample ZS-1 zeolites were prepared by hydrothermal crystallization from gels consisting of a Si source, a Zn source, and HMI as the template. Several samples have been prepared with different Si/Zn ratios as well as sodium contents.

It is expected that variation of the Si/Zn ratio over a wide range would allow adjustment of the catalytic properties of the resulting zeolites to achieve a desired performance.

In a typical process for preparation of the sample catalyst, 1.06 g sodium hydroxide and 3.52 g zinc nitrate hexahydrate were mixed with 86.7 g of distilled water until the reagents were completely dissolved. Next, 5.44 g of hexamethyleneimine was added to the mixture and stirred for 10 min. After that, 18.31 g Ludox HS-40 colloidal silica and 0.11 g of MCM-22 seeds were added to the mixture, which was continuously stirred for about 4 hours until a gel was formed. The resulting gel solution, with a pH above 7, was transferred to an autoclave and heated to allow the gel to crystallize at 150° C. for 2 to 6 weeks in a rotary oven. The product in the autoclave contained a slurry. The slurry was removed from the oven, and was cooled, filtered and washed thoroughly with distilled water and then dried at 80° C. to 110° C. for several hours. The dried material was calcined in a static air furnace at 550° C. for 7 to 12 hours to form the sample zeolite materials.

The sample zeolites were found to be pure Zn—Si zeolites with MWW framework. Representative XRD patterns taken from sample ZS-1 zeolites are shown in FIGS. 1A and 1B. The characteristic XRD peaks of the sample ZS-1 shown in FIGS. 1A, 1B are also listed in Table III.

TABLE III
XRD peaks of Sample ZS-1 Zeolite

| Peak (d-value) | 2-θ (degree) | Intensity (counts/s) |
|---|---|---|
| 27.29169 | 3.235 | 3114 |
| 12.35172 | 7.151 | 6924 |
| 11.07597 | 7.976 | 4782 |
| 8.84895 | 9.988 | 3674 |
| 6.86295 | 12.889 | 1897 |
| 6.17105 | 14.341 | 2829 |
| 6.02323 | 14.695 | 1591 |
| 5.53913 | 15.988 | 1559 |
| 4.65454 | 19.052 | 1060 |
| 4.38194 | 20.249 | 1204 |
| 4.09719 | 21.673 | 1489 |
| 4.04913 | 21.933 | 1620 |
| 3.90646 | 22.745 | 2092 |
| 3.74614 | 23.732 | 1404 |
| 3.55397 | 25.036 | 1371 |
| 3.41576 | 26.066 | 3397 |
| 3.29766 | 27.017 | 1332 |
| 3.20208 | 27.840 | 1175 |
| 2.82189 | 31.683 | 805 |
| 2.67625 | 33.456 | 966 |
| 2.37085 | 37.920 | 674 |

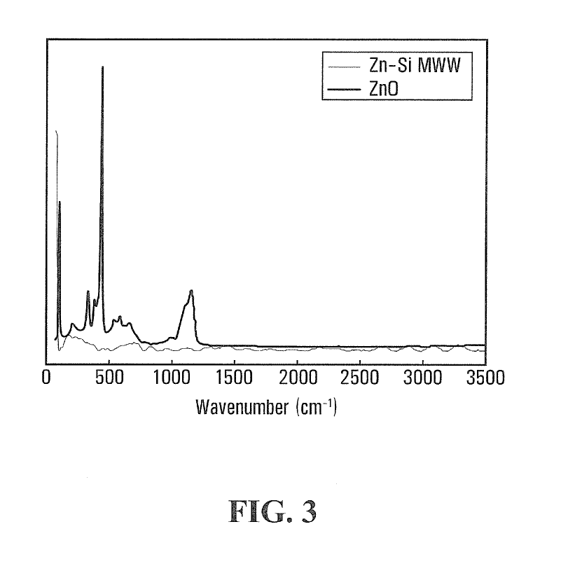

FIG. 2 shows a representative comparison of the FTIR spectra of a sample ZS-1 zeolite and comparison materials (MCM-22 and ZnO). FIG. 3 shows comparison of Roman spectroscopy spectra of the sample ZS-1 zeolite and ZnO.

The comparison with MCM-22 and ZnO indicated that the form of Zn in the sample ZS-1 zeolite was different from the form of Zn in the ZnO zeolite. The shift of the IR (infrared resonance) band from 817 cm^{-I} (in the MCM-22) to a lower wavenumber (in the ZS-1 zeolite) suggested that Zn in the sample Zs-1 zeolite was in the MWW framework.

### Example II

### Synthesis and Characterization of Sample Fe—Si Zeolite

Sample Fe—Si zeolites with MWW framework were prepared by hydrothermal crystallization from gels consisting of a Si source, a Fe source, and HMI as the template.

In a typical preparation process, 1.06 g sodium hydroxide and 4.78 g iron nitrate hydrate were mixed with 86.7 g of distilled water until the reagents were completely dissolved. Next, 5.44 g of hexamethyleneimine was added to the mixture and stirred for 10 min. After that 18.31 g Ludox HS-40 colloidal silica and 0.11 g of MCM-22 seeds were added to the mixture, which was continuously stirred for about 4 hours until a gel was formed. The resulting gel solution, with a pH above 7, was transferred to an autoclave and heated to allow the gel to crystallize at 150° C. for 2 to 6 weeks in a rotary oven. The product in the autoclave contained a slurry. The slurry was removed from the oven, and was cooled, filtered and washed thoroughly with distilled water and dried at 80° C. to 110° C. for several hours. The dried material was calcined in a static air furnace at 550° C. for 7 to 12 hours to form a sample zeolite material.

The sample zeolites prepared were found to be pure Fe—Si zeolites with MWW framework. A representative XRD spectrum taken from a sample Fe—Si zeolite is shown in FIG. 4. The characteristic XRD peaks of a sample Fe—Si zeolite are also listed in Table IV.

TABLE IV
XRD peaks of Sample Fe—Si Zeolite

| Peak (d-value) | 2-θ (degree) | Intensity (counts/s) |
|---|---|---|
| 11.19660 | 7.89 | 1626 |
| 10.06619 | 8.778 | 1225 |
| 6.86684 | 12.882 | 783 |
| 5.84501 | 15.146 | 692 |
| 4.63922 | 19.115 | 904 |
| 4.33249 | 20.483 | 1185 |
| 3.99949 | 22.209 | 840 |
| 3.85260 | 23.067 | 706 |
| 3.82832 | 23.216 | 744 |
| 3.70707 | 23.986 | 669 |
| 3.35340 | 26.560 | 644 |
| 3.27486 | 27.209 | 857 |

### Example III

### Ethanol Dehydrogenation

It was demonstrated that sample ZS-1 zeolites exhibited good catalytic activity for direct ethanol dehydrogenation. Tests were conducted using ZS-1 zeolites as sample catalysts.

The test reactions were conducted in a continuous fixed-bed reactor. Typically, 500 mg of a sample ZS-1 catalyst was loaded in the reactor. Prior to the reaction, the sample catalyst was pre-treated in 5% argon/helium (100 mL/min) at 450° C. for one hour. Ethanol was then fed to the reactor at a space velocity of 9.5 h⁻¹. The reaction products were analyzed with an on-line gas chromatograph (Agilent, 6890N) equipped with both flame ionization detector (FID) and thermal conductivity detector (TCD).

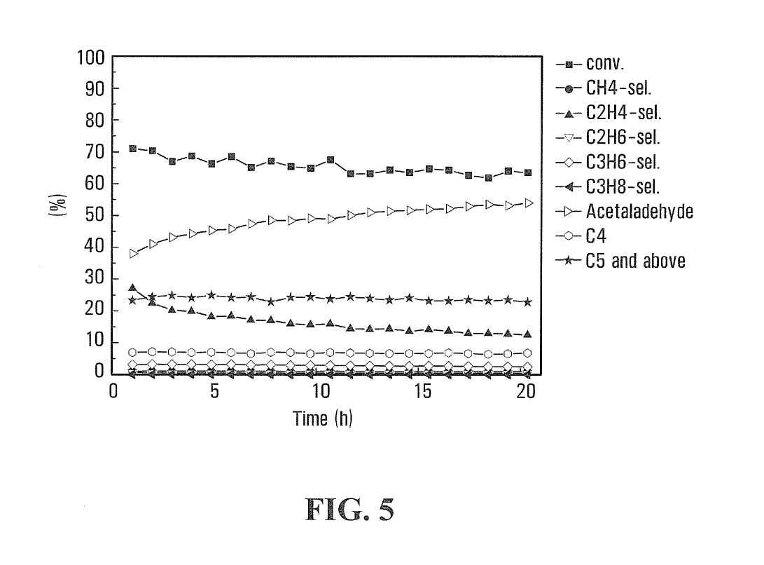

Representative catalytic results are shown in FIG. 5, which were obtained under the following reaction conditions: the amount of the sample catalyst 0.5 g; Ethanol GHSV 9.5 h⁻¹; Temperature 400° C.; Pressure 0.1 MPa.

As shown in FIG. 5, acetaldehyde was the main product. The selectivity increased with the reaction time, and reached above 50% acetaldehyde selectivity after 20 hours of reaction time. From the test results, it can be expected that ZS-1 zeolite materials could directly dehydrogenate ethanol without requiring molecular oxygen. It may also be expected that the yield of acetaldehyde could be higher with optimization of the process.

### Example IV

### Butene Oxidative Dehydrogenation

It was also demonstrated that ZS-1 zeolite could exhibit good catalytic activity for butene oxidative dehydrogenation with CO₂ as soft oxidant.

The testing reactions were conducted in a continuous fixed-bed reactor. Typically, 100 mg of a sample ZS-1 catalyst was loaded in the reactor. Prior to the reaction, the sample catalyst was pre-treated in argon (30 mL/min) at 400° C. for one hour. The reactant gas mixture was fed into the reactor with a molar ratio of 1-butene/CO₂ at 1:9 and a total flow of 30 mL/min (WHSV=4.5 h⁻¹). The reaction products were analyzed with an on-line gas chromatograph (Agilent, 6890N) equipped with both flame ionization detector (FID) and thermal conductivity detector (TCD).

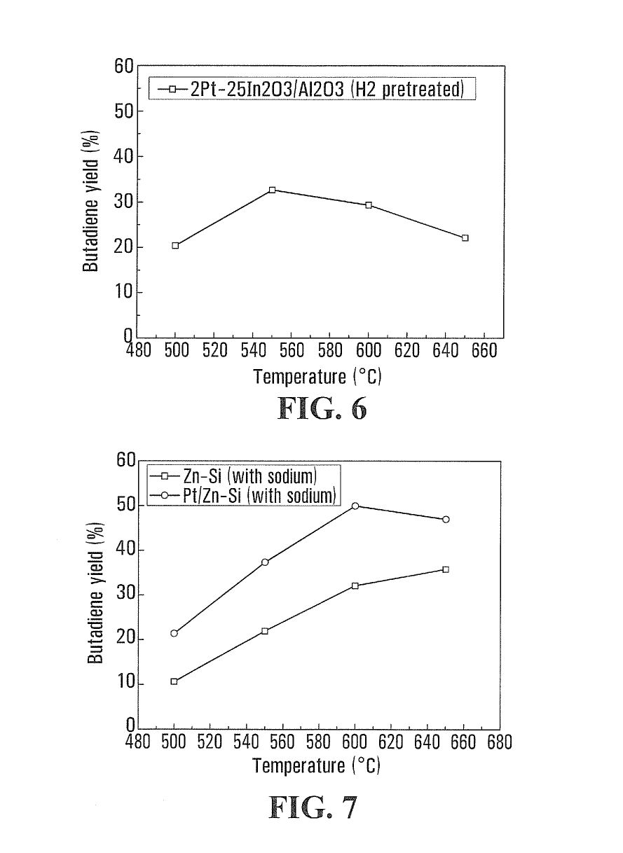

Representative of the test results are shown in FIG. 6, which shows butene dehydrogenation activity of sample ZS-1 zeolite with or without Pt. When the samples were loaded with Pt, the Pt loadings varied from 0.5% to 5%, and were loaded by an impregnation method. The reaction conditions were as follows: catalyst 0.10 g; butene:CO₂ 1:9; flowrate: 30 ml/min; pressure 0.1 MPa.

It is expected that the noble metal, Pt, could further improve butadiene yield up to 50%, much higher than the yields obtained on transition metal oxide supported on alumina support (30% at the same reaction condition).

Representative comparison results are shown in FIG. 7, which show catalytic activity of Pt—In₂O₃/Al₂O₃ at the same conditions as for FIG. 6.

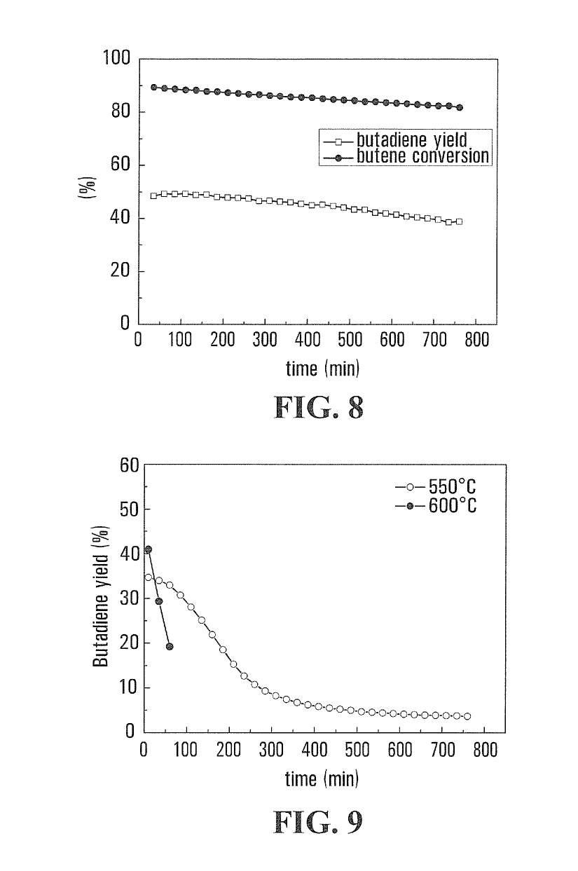

The test results showed that not only the butadiene yield but also the catalytic stability was superior for the Pt/Zn—Si MWW sample, as can be seen in FIG. 8.

FIG. 8 shows representative stability test results for Pt/ZS-1 zeolite, obtained under the following conditions: catalyst 0.10 g; butene:CO₂ 1:9; flowrate: 30 ml/min; temperature 600° C.; pressure 0.1 MPa. As shown in FIG. 8, the butadiene yield and butene conversion were kept more or less stable up to 13 hours.

FIG. 9 shows stability testing of Pt—In₂O₃/Al₂O₃ at the same conditions as for FIG. 8. It can be seen that the stability for sample ZS-1 zeolite was significantly better for the transition metal oxide supported on an alumina support.

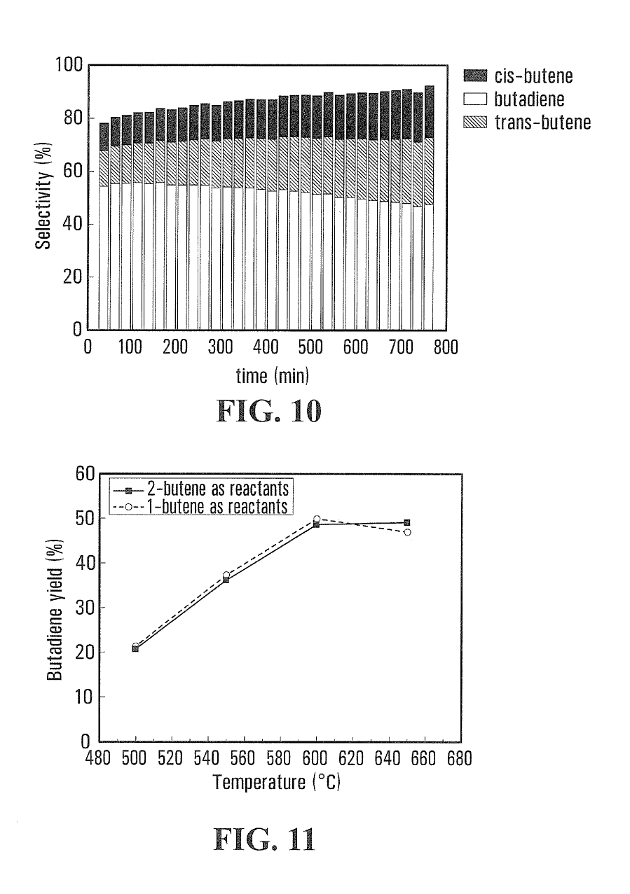

It was observed that the by-products of the reaction were trans- and cis-2-butene. Altogether, the total selectivity of these three products was more than 80%, as can be seen in FIG. 10, which shows distribution of product selectivity for these three products when a Pt/ZS-1 zeolite was used as the catalyst at the following conditions: catalyst 0.10 g; butene:CO₂ 1:9; flowrate: 30 ml/min; pressure 0.1 MPa. The results indicated that the selectivity to lighter or heavier molecules than C4 was rather low.

Tests also showed that trans- and cis-2-butene could be converted to 1,3-butadiene with a similar yield. Representative results are shown in FIG. 11, which compares catalytic activity using either 1-butene or 2-butene feedstock, at the following reaction conditions: catalyst 0.10g; butene or trans/cis-2-butene:CO₂ 1:9; flowrate: 30 ml/min; pressure 0.1 MPa. The test results suggested that trans/cis-2-butene produced in the oxidative dehydrogenation reaction of 1-butene could be recycled to further produce butadiene.

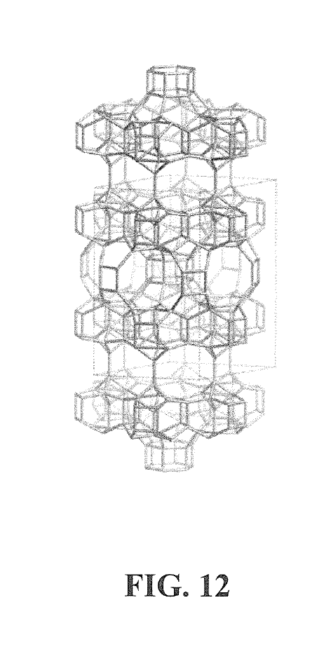

As in known MCM-22 zeolites, the MWW frameworks in the sample ZS-1 zeolites or Fe—Si zeolites were expected to have a layered structure and two pore systems as illustrated by the schematic representation shown in FIG. 12.

### CONCLUDING REMARKS

It will be understood that any range of values herein is intended to specifically include any intermediate value or sub-range within the given range, and all such intermediate values and sub-ranges are individually and specifically disclosed.

It will also be understood that the word “a” or “an” is intended to mean “one or more” or “at least one”, and any singular form is intended to include plurals herein.

It will be further understood that the term “comprise”, including any variation thereof, is intended to be open-ended and means “include, but not limited to,” unless otherwise specifically indicated to the contrary.

When a list of items is given herein with an “or” before the last item, any one of the listed items or any suitable combination of two or more of the listed items may be selected and used.

Of course, the above described embodiments of the invention are intended to be illustrative only and in no way limiting. The described embodiments of the invention are susceptible to many modifications of form, arrangement of parts, details and order of operation. The invention, rather, is intended to encompass all such modification within its scope, as defined by the claims.

1. A method comprising:
treating a gel solution by a hydrothermal treatment, to form a Zn—Si or Fe—Si zeolite having an MWW framework, wherein said gel solution comprises water, a silicon source, a zinc or iron source, and an MWW-templating agent,
wherein a molar ratio of said silicon source to said zinc or iron source in said gel solution is from 7:1 to 20:1; or wherein said gel solution comprises about 20 wt % of said silicon source and about 3 to about 4 wt % of said zinc or iron source, based on a total weight of said gel solution, and wherein said MWW framework is substantially free of aluminum.

2. The method of claim 1, wherein said gel solution is maintained at a first temperature to dissolve said silicon source and said zinc or iron source in said gel solution; and said hydrothermal treatment comprises heating the gel solution with the dissolved silicon source and zinc or iron source at a second temperature above the first temperature, to induce hydrothermal crystallization in said gel solution, thus forming a crystalline Zn—Si or Fe—Si zeolite having said MWW framework.

3. The method of claim 2, wherein said first temperature is in the range from room temperature to about 70° C.; and said second temperature is in the range from about 140° C. to about 160° C.

4. The method of claim 2, wherein said gel solution is heated at said first temperature for a period of up to about 24 hours, and at said second temperature for a period of 1 to 6 weeks.

5. The method of claim 1, wherein said MWW-templating agent comprises a hexamethyleneimine.

6. The method of claim 1, wherein said silicon source is selected from colloidal silica, fumed silica, silicon oxides, silicates, and mixtures thereof; and wherein said zinc or iron source is selected from zinc nitrates, zinc halides, zinc sulfates, zinc carbonates, zinc alkoxides, and mixtures thereof.

7. The method of claim 1, wherein said gel solution comprises a base.

8. The method of claim 7, wherein said base comprises sodium hydroxide.

9. The method of claim 1, wherein said gel solution has a pH value of above 7 before said hydrothermal treatment.

10. The method of claim 1, wherein said gel solution comprises seeds having an MWW framework.

11. The method of claim 1, wherein said Zn—Si or Fe—Si zeolite is a Zn—Si zeolite, and said zinc or iron source is a zinc source.

12. A zeolite prepared according to a method comprising:
treating a gel solution by a hydrothermal treatment, to form a Zn—Si or Fe—Si zeolite having an MWW framework, wherein said gel solution comprises water, a silicon source, a zinc or iron source, and an MWW-templating agent,
wherein a molar ratio of said silicon source to said zinc or iron source in said gel solution is from 7:1 to 20:1; or wherein said gel solution comprises about 20 wt % of said silicon source and about 3 to about 4 wt % of said zinc or iron source, based on a total weight of said gel solution, and
wherein said MWW framework is substantially free of aluminum.

13. A Zn—Si zeolite having an MWW framework substantially free of aluminum.

14. The zeolite of claim 12, characterized by an x-ray diffraction pattern comprising the following peaks:

| d-value (Å) | 2-θ (degree) | Relative Intensity |
|---|---|---|
| 12.4 | 7.2 | High |
| 11.1 | 8.0 | High |
| 8.85 | 10.0 | Medium |
| 6.17 | 14.3 | Medium |
| 3.91 | 22.7 | Medium |
| 3.42 | 26.1 | Medium |

15. A Fe—Si zeolite having an MWW framework substantially free of aluminum.

16. The zeolite of claim 12, characterized by an x-ray diffraction pattern comprising the following peaks:

| d-value (Å) | 2-θ (degree) | Relative Intensity |
|---|---|---|
| 11.2 | 7.9 | High |
| 10.1 | 8.8 | Medium |
| 6.87 | 12.9 | Low |
| 5.85 | 15.1 | Low |
| 4.00 | 22.2 | Medium |
| 3.35 | 26.6 | Low |

17. A catalyst comprising a zeolite prepared according to a method comprising:
treating a gel solution by a hydrothermal treatment, to form a Zn—Si or Fe—Si zeolite having an MWW framework, wherein said gel solution comprises water, a silicon source, a zinc or iron source, and an MWW-templating agent,
wherein a molar ratio of said silicon source to said zinc or iron source in said gel solution is from 7:1 to 20:1; or wherein said gel solution comprises about 20 wt % of said silicon source and about 3 to about 4 wt % of said zinc or iron source, based on a total weight of said gel solution, and
wherein said MWW framework is substantially free of aluminum.

18. The catalyst of claim 17 for use in catalyzing an activation, oxidehydrogenation, or dehydrogenation reaction.

19. A separation or purification device comprising a zeolite prepared according to a method comprising:
treating a gel solution by a hydrothermal treatment, to form a Zn—Si or Fe—Si zeolite having an MWW framework, wherein said gel solution comprises water, a silicon source, a zinc or iron source, and an MWW-templating agent,
wherein a molar ratio of said silicon source to said zinc or iron source in said gel solution is from 7:1 to 20:1; or wherein said gel solution comprises about 20 wt % of said silicon source and about 3 to about 4 wt % of said zinc or iron source, based on a total weight of said gel solution, and wherein said MWW framework is substantially free of aluminum.
